(19) United States
(12) Patent Application Publication
(10) Pub. No.: US 2005/0118246 A1
(43) Pub. Date: Jun. 2, 2005

(54) Dosage forms and layered deposition processes for fabricating dosage forms

(76) Inventors: Patrick S.L. Wong, Burlingame, CA (US); B. Michael Silber, Palo Alto, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/976,589

(22) Filed: Oct. 29, 2004

Related U.S. Application Data
(60) Provisional application No. 60/516,251, filed on Oct. 31, 2003.

(51) Int. Cl. A61K 9/70; A61K 9/14

(52) U.S. Cl. 424/449; 424/488

(57) ABSTRACT

Disclosed are transdermal or transmucosal dosage forms which include a matrix and a drug where the total amount of drug present in the dosage form exceeds the solubility limit of the drug in the matrix. Also disclosed are transdermal or transmucosal dosage forms which include two or more drug-containing layers and one or more intervening hydrophilic layers where the two or more drug-containing layers being separated from one another by the one or more intervening hydrophilic layers. Methods for delaying release and delivery of an active from an active layer disposed in a transdermal or transmucosal dosage form are also disclosed, as well as methods for manufacturing transdermal or transmucosal dosage forms by providing a substrate and disposing at least one transdermal or transmucosal dosage form layer on the substrate using a printing process.

### CROSS-REFERENCES TO RELATED APPLICATIONS

This application claims the benefit of U.S. Provisional Application No. 60/516,251, filed on Oct. 31, 2003.

### FIELD OF THE INVENTION

The present invention is directed, generally, to dosage forms and, more particularly, to dosage forms suitable for transdermal and/or transmucosal delivery of active agents.

### BACKGROUND OF THE INVENTION

Transdermal and transmucosal delivery systems have been employed to deliver a number of active agents to a variety of subjects. However, in conventional transdermal systems in which the active agent is dispersed in a matrix, active agents can only be delivered with a single profile (e.g., a descending or zero-order profile). Moreover, since the initial drug loading into the matrix is limited to the solubility limit of the active agent in the material from which the matrix is made, in practice, as much as fifty percent of the active agent initially loaded into the matrix remains in the matrix after use. Higher loadings of active agent (i.e., loadings which exceed the solubility limit for the active agent in the matrix) can be problematic, for example, due to stabilization and dispersion issues within the matrix. The present invention, in part, is directed to overcoming at least some of these shortcomings in conventional transdermal systems.

### SUMMARY OF THE INVENTION

The present invention relates to a transdermal or transmucosal dosage form which includes a matrix and a drug in which the total amount of drug present in the dosage form exceeds the solubility limit of the drug in the matrix.

The present invention also relates to a transdermal or transmucosal dosage form which includes two or more drug-containing layers and one or more intervening hydrophilic layers, the two or more drug-containing layers being separated from one another by the one or more intervening hydrophilic layers.

The present invention also relates to a method for preparing a transdermal or transmucosal dosage form which includes a matrix and a drug dispersed in the matrix, where the total amount of the drug present in the dosage form exceeds the solubility limit of the drug in the matrix. The method includes sequentially forming two or more layers of drug and two or more layers of matrix such that the amount of drug contained in the two or more layers of drug exceeds the solubility limit of the drug in the matrix.

The present invention also relates to a method for delaying release of an active from an active layer disposed in a transdermal or transmucosal dosage form which dosage form includes, in addition to the active layer, an adhesive layer. The method includes disposing one or more hydrophilic layers between the adhesive layer and the active layer.

The present invention also relates to a method for delaying delivery of an active from an active layer disposed in a transdermal or transmucosal dosage form to a subject's skin or mucosa. The method includes disposing, in the dosage form, one or more hydrophilic layers between the active layer and the subject's skin or mucosa.

The present invention also relates to a method of manufacturing a transdermal or transmucosal dosage form. The method includes providing a substrate and disposing at least one transdermal or transmucosal dosage form layer on the substrate using a printing process.

### BRIEF DESCRIPTION OF THE DRAWINGS

FIGS. 1A-1E are cross-sectional views of various dosage forms in accordance with the present invention.

FIG. 2 is a cross-sectional view of a dosage form in accordance with the present invention.

FIGS. 3A and 3B are cross-sectional views of dosage forms in accordance with the present invention. FIG. 3C is an illustrative drug release profile showing drug release rate as a function of time for a dosage form of the present invention.

FIGS. 4A-4C are perspective views of various layer set configurations which can be employed in dosage forms of the present invention.

FIG. 5A is a cross-sectional view of a dosage form in accordance with the present invention. FIG. 5B is an illustrative drug release profile showing drug release rate as a function of time for a dosage form in accordance with the present invention.

FIGS. 6A-6C are graphs of drug release rate as a function of time illustrating various drug release profiles for a various dosage forms of the present invention.

FIGS. 7A and 7B are cross-sectional views of dosage forms in accordance with the present invention. FIG. 7C is an illustrative drug release profile showing drug release rate as a function of time for a dosage form of the present invention.

### DETAILED DESCRIPTION OF THE INVENTION

The present invention, in one aspect thereof, relates to a transdermal or transmucosal dosage form which includes a matrix and a drug in which the total amount of drug present in the dosage form exceeds the solubility limit of the drug in the matrix.

“Transdermal dosage form”, as used herein, is meant to include a device for controlled delivery of one or more drugs to a subject's skin. “Transmucosal dosage form”, as used herein, is meant to include a device for controlled delivery of one or more drugs to a mucous membrane of a subject, such as a mucous membrane in the subject's vaginal cavity, in the subject's nasal cavity or other airway, or in the subject's oral cavity, esophagus, stomach, small intestine, large intestine, rectum, or other portion of the gastrointestinal tract. The transdermal and transmucosal dosage forms can be of any suitable size and shape. Illustratively, they can be circular, square, or rectangular disks having an area of from about 0.5 mm² to about 50 cm², such as from about 1 mm² to about 15 cm². For example, in applications where the dosage form is to be administered to the gut, suitable dosage forms include those having areas of from about 0.5 mm² to about 20 mm², such as from about 1 mm² to about 10 mm², as well as circular disks having diameters of from about 1 mm to about 4 mm. Suitable aspect ratios range from about 1:5 (total disk thickness:disk diameter) to about 1:100, such as from about 1:10 to about 1:50.

“Subject”, as used herein is meant to include, for example, mammals, such as humans and other primates; dogs and other canines; cats and other felines; horses and other equines; cows and other bovines; pigs and other porcines; mice, rats, and other rodents; sheep, goats, and the like.

“Drug”, as used herein, is meant to include any biologically active material, such as, for example, nicotine; corticosteroids, such as hydrocortisone, prednisolone, beclomethasone-propionate, flumethasone, triamcinolone, triamcinolone-acetonide, fluocinolon, fluocinolinacetonide, fluocinolon-acetonide acetate, clobetasolpropionate, etc.; analgesics and/or anti-inflammatory agents, such as acetaminophen, mefenamic acid, flufenamic acid, diclofenac, diclofenac-sodium-alclofenac, oxyphenbutazone, phenylbutazone, ibuprofen, flurbiprofen, salicylic acid, 1-menthol, camphor, sulindac-tolmetin-sodium, naproxen, fenbufen, etc.; hypnotically active sedatives, such as phenobarbital, amobarbital, cyclobarbital, triazolam, nitrazepam, lorazepam, haloperidol, etc.; tranquilizers, such as fluphenazine, thioridazine, lorazepam, flunitrazepam, chloropromazine, etc.; antihypertensives, such as pindolol, indenolol, nifedipin, lofexidin, nipradinol, bucumolol, etc.; antihypertensively acting diuretics, such as hydrothiazide, bendroflumethiazide, cyclopenthiazide, etc.; antibiotics, such as penicillin, tetracycline, oxytetracycline, fradiomycin suflate, erythromycin, chloramphenicol, etc.; anesthetics, such as lidocaine, benzocaine, ethylaminobenzoate, etc.; antimicrobiological agents, such as benzalkonium chloride, nitrofurazone, nystatin, acetosulfamine, clotrimazole, etc.; antifungal agents, such as pentamycin, amphotericin B, pyrrolnitrin, clotrimazole, etc.; vitamins, such as vitamin A, ergocalciferol, chlolecalciferol, octotiamine, riboflavin butyrate, etc.; antiepileptics, such as nitrazepam, meprobamate, clonazepam, etc.; coronary vasodilators, such as dipyridamole, erythritol tetranitrate, pentaerythritol tetranitrate, propatylnitrate, etc.; antihistamines, such as diphenyl hydromine hydrochloride, chlorpheniramine, diphenylimidazole, etc.; antitussives, such as dertromethorphan (hydrobromide), terbutaline (sulphate), ephedrine (hydrochloride), salbutanol (sulphate), isoproterenol (sulfate, hydrochloride), etc.; sexual hormones, such as progesterone, etc.; thymoleptics, such as doxepin, etc.; narcotic analgesics, opioid analgesics, and/or other types of analgesics, such as alfentanil, allylprodine, alphaprodine, anileridine, benzylmorphine, bezitramide, buprenorphine, butorphanol, clonitazene, codeine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, hydrocodone, hydromorphone, hydroxypethidine, isomethadone, ketobemidone, levorphanol, lofentanil, meperidine, meptazionl, metazocine, methadone, metopon, morphine, myrophine, nalbuphine, narceine, nicomorphine, norlevorphanol, normethadone, normorphine, norpipanone, opium, oxycodone, oxymorphone, papaveretum, pentazocine, phenadoxone, phenazocine, phenoperidine, piminodine, piritramide, proheptazine, promedol, properidine, propiram, propoxyphene, sufentanil, tilidine; pharmaceutically acceptable salts thereof; as well as combinations thereof.

Matrix, as used herein, is meant to include, for example, any structural polymer, such as mixed or partially substituted hydroxyalkyl ethers of cellulose, polyisobutylene, polybutadiene, ethylenevinyl acetate copolymers, poly(vinyl alcohol), poly(vinyl pyrrolidone), and the like.

As one skilled in the art will appreciate, each drug will have a solubility limit in a particular matrix, i.e., the concentration of drug which, when exceeded, results in excess drug being present in a non-solubilized form (e.g., in the form of a dispersion and/or in the form of particles). Solubility limits for various drug/matrix combinations are known in the art. For other drug/matrix combinations, the solubility limit can be determined by methods well known to those skilled in the art.

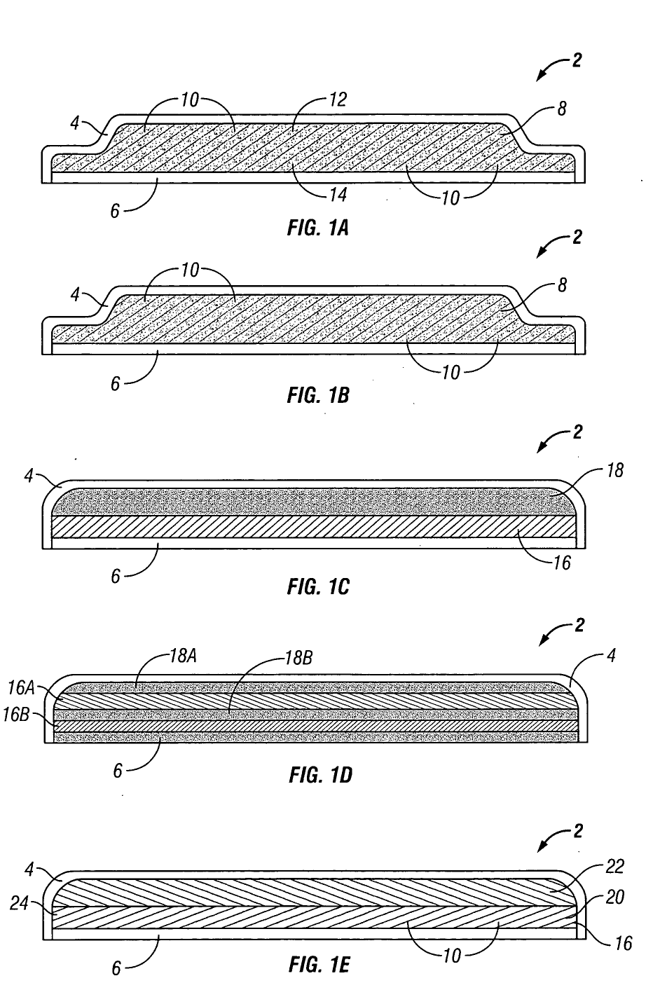

In one embodiment of the present invention, the drug is dispersed in the matrix at a concentration greater than the solubility limit of the drug in the matrix. This is illustrated in FIGS. 1A and 1B. Referring to FIGS. 1A and 1B, dosage form 2 includes backing layer 4 and adhesive layer 6 which together enclose matrix 8. Matrix 8 contains drug 10. In FIG. 1A, the concentration of drug 10 in upper portion 12 of matrix 8 is shown as being greater than the concentration of drug 10 in lower portion 14 of matrix 8. Alternatively, drug 10 can be substantially uniformly dispersed in matrix 8, as illustrated in FIG. 1B. Still alternatively, the concentration of the drug in the matrix can vary in a substantially continuous manner or in a stepwise manner (e.g., to form a substantially continuous gradient or a stepwise gradient), with the lowest concentration being in the lower portion of the matrix (i.e., in the portion closest to the adhesive layer, farthest from the backing layer, or most proximal to the skin or mucosa to which the dosage form is to be adhered) and with the highest concentration being in the upper portion of the matrix (i.e., in the portion farthest from the adhesive layer, closest to the backing layer, or most distal to the skin or mucosa to which the dosage form is to be adhered). Still alternatively, the concentration of the drug in the matrix can vary in a substantially continuous manner or in a stepwise manner (e.g., to form a substantially continuous gradient or a stepwise gradient), with the highest concentration being in the lower portion of the matrix (i.e., in the portion closest to the adhesive layer, farthest from the backing layer, or most proximal to the skin or mucosa to which the dosage form is to be adhered) and the lowest concentration being in the upper portion of the matrix (i.e., in the portion farthest from the adhesive layer, closest to the backing layer, or most distal to the skin or mucosa to which the dosage form is to be adhered). Still alternatively or additionally, the concentration of the drug in the matrix can vary in a discontinuous manner such that the concentration of drug in the matrix varies between a series of maxima and minima, the distance between at least two adjacent maxima being less than 4 microns, such as less than about 4 microns, less than about 3 microns, less than about 2 microns, less than about 1 micron, and/or less than about 100 nm. In each of the above-described embodiments, the drug can be present as individual molecules, agglomerated molecules, particles having a variety of dimensions, particles of substantially equal dimension, or combinations of these, as in the case where some of the drug is present as individual molecules and some of the drug is present as agglomerated molecules, particles having a variety of dimensions, and/or particles of substantially equal dimension.

In another embodiment, the drug and the matrix are present as substantially discrete layers. This is illustrated in FIGS. 1C and 1D. Referring to FIG. 1C, dosage form 2 includes backing layer 4 and adhesive layer 6 which together enclose matrix layer 16 and drug layer 18. Referring to FIG. 1D, dosage form 2 includes backing layer 4 and adhesive layer 6 which together enclose a plurality of matrix layers 16a and 16b and a plurality of drug layers 18a and 18b.

Still alternatively, a portion of the drug contained in the dosage form can be present as a discrete layer and a portion of the drug contained in the dosage form can be dispersed in the matrix. In such embodiments, the portion of drug which is dispersed in the matrix can be present at a concentration greater than the solubility limit of the drug in the matrix, at a concentration equal to the solubility limit of the drug in the matrix, or at a concentration lower than the solubility limit of the drug in the matrix. The portion of drug which is dispersed in the matrix can be, but need not be, substantially uniformly dispersed in the matrix. FIG. 1E illustrates one embodiment in which a portion of the drug contained in the dosage form is present as a discrete layer and a portion of the drug contained in the dosage form is dispersed in the matrix. Referring to FIG. 1E, dosage form 2 includes backing layer 4 and adhesive layer 6 which together enclose matrix layer 20 and drug layer 22. Upper portion 24 of matrix layer 20 contains drug 10 which can be present as individual molecules, agglomerated molecules, particles having a variety of dimensions, particles of substantially equal dimension, or combinations of these, as in the case where some of drug 10 is present as individual molecules and some of drug 10 is present as agglomerated molecules, particles having a variety of dimensions, and/or particles of substantially equal dimension.

In those embodiments in which at least a portion of the drug contained in the dosage form is present as a discrete layer or as discrete layers, the drug-containing layer or layers can contain only drug, or the drug-containing layer or layers can contain, in addition to the drug, other components, such as matrix (provided that the concentration of drug in the drug-containing layer is sufficiently greater than the concentration of drug in the matrix layer such that the layers are distinguishable), permeation enhancers, stabilizers, antioxidants, excipients, anti-irritants, and anti-inflammatory materials (e.g., steroids and/or other anti-inflammatory materials, for example, to reduce redness).

In those embodiments in which at least a portion of the drug contained in the dosage form is present as a discrete layer or as discrete layers, the thickness of the drug-containing layer or layers can range from about 1 nm to about 10 micron, such as from about 10 nm to about 100 nm, from about 100 nm to about 1 micron, and/or from about 100 nm to about 10 micron; and the thickness of the matrix layers can range from about 100 nm to about 100 micron, such as from about 1 micron to about 10 micron.

In those embodiments in which at least a portion of the drug contained in the dosage form is present as a plurality of discrete layers alternating with a plurality of matrix layers, the number of drug layer/matrix layer pairs can range from 2 to about 1000, such as from about 5 to about 100, from about 5 to about 50, from about 5 to about 20, etc. The amount of drug present in each of the matrix layers can be the same, or it can vary, for example, as in those cases where the concentration of the drug in the matrix layers varies in a stepwise manner, with the lowest concentration being in the lower matrix layer (i.e., in the matrix layer closest to the adhesive layer, farthest from the backing layer, or most proximal to the skin or mucosa to which the dosage form is to be adhered) and with the highest concentration being in the upper matrix layer (i.e., in the matrix layer farthest from the adhesive layer, closest to the backing layer, or most distal to the skin or mucosa to which the dosage form is to be adhered).

In still other embodiments, the matrix can be present as a plurality of matrix layers, which can be distinguished from one another by (i) the nature of the matrix, (ii) by the nature of the drug, or (iii) by both the nature of the matrix and by the nature of the drug. For example, in one such embodiment, the matrix is present as a plurality of matrix layers where the matrix layers are the same or different and where no two adjacent matrix layers contain the same drug, such as in the case (i) where Drug A is present in the first matrix layer, Drug B is present in the second matrix layer, Drug A is present in the third matrix layer, Drug B is present in the fourth matrix layer, etc. or (ii) where Drug A is present in the first matrix layer, Drug B is present in the second matrix layer, Drug C is present in the third matrix layer, Drug A is present in the fourth matrix layer, etc. As a further example, in another such embodiment, the matrix is present as a plurality of matrix layers where each matrix layer contains the same drug in the same of different concentrations and where no two adjacent matrix layers are made from the same matrix material, such as in the case (i) where the matrix is constructed from alternating layers of Matrix A and Matrix B, where Matrix A and Matrix B have different properties, e.g., different solubility limits for the drug, different drug diffusion characteristics, etc.; (ii) where the matrix is constructed from layers of Matrix A and Matrix B, Matrix A having a lower solubility limit for the drug than Matrix B and Matrix A being closer to the skin or mucosa than Matrix B; or (iii) where the matrix is constructed from a plurality of matrix layers (e.g., Matrix A, Matrix B, and Matrix C) where the matrix layers are arranged such that the matrix layers closer to the skin have a solubility limit for the drug which is lower than the solubility limit for the drug of the matrix layers more distal from the skin or mucosa (e.g., where the layers are arranged with Matrix A being closest to the skin or mucosa, with Matrix B being adjacent to Matrix A and Matrix C being adjacent to Matrix B and where the solubility limit of the drug in Matrix A is lower than the solubility limit of the drug in Matrix B and the solubility limit of the drug in Matrix B is lower than the solubility limit of the drug in Matrix C). It will be appreciated that, in any given matrix layer, the drug can be substantially uniformly distributed or not. It will be further appreciated that the matrix layers can have same thickness or different thickness, for example, as in the case where the matrix layers have the same or different thicknesses, each being less than about 4 micron, such as less than about 3 micron, less than about 2 micron, less than about 1 microns, less than about 100 nm, etc. and/or as in the case as where at least one of the matrix layers has thickness of less than about 4 micron, such as less than about 3 micron, less than about 2 micron, less than about 1 microns, less than about 100 nm, etc.

In still further embodiments, the matrix can be present as an adhesive layer (discussed further below), such as a mucoadhesive layer, or, in cases where a plurality of matrix layers are employed, one of the matrix layers can be present as the adhesive layer.

The dosage form can include other components (e.g., in addition to the drug and matrix).

For example, as discussed above, the dosage form can include a backing layer. The backing layer is generally selected so as to be impermeable to the drug contained in the dosage form and so as to seal the dosage form from the external environment. The backing layer can comprise, for example, a polymer which is insoluble in and impermeable to aqueous media, such as polyolefins, polyesters, acrylonitriles, polyethyinaphthalenes, polyethylene terephthalates, polyimides, polyurethanes, polyethylenes, metallized or glass-coated ethylyne copolymer films (e.g., metallized or glass-coated ethylene-vinyl acetate copolymer films), or combinations thereof. The backing layer can have a thickness of from about 0.3 mil to about 10 mil, such as from about 0.5 mil to about 5 mil, from about 1 mil to about 4 mil, from about 1.5 mil to about 3.5 mil, and/or from about 1 mil to about 2 mil.

Also as discussed above, the dosage form can include an adhesive layer. The adhesive layer can comprise an adhesive such as a pressure sensitive adhesive, such as, for example, polyacrylates, polysiloxanes, polyisobutylene, polyisoprene, polybutadiene, styrenic block polymers, and the like.

Examples of suitable styrenic block copolymer-based adhesives include styrene-isoprene-styrene (“SIS”) block copolymer, styrene-butadiene-styrene (“SBS”) block copolymer, styrene-ethylynebutene-styrene (“SEBS”) block copolymer, and di-block analogs thereof.

Examples acrylic polymer-based adhesives include those which are comprised of a copolymer or terpolymer comprising at least two or more exemplary components selected from the group comprising acrylic acids, alkyl acrylates, methacrylates, copolymerizable secondary monomers or monomers with functional groups. Examples of monomers include, but are not limited to, acrylic acid, methacrylic acid, methoxyethyl acrylate, ethyl acrylate, butyl acrylate, butyl methacrylate, hexyl acrylate, hexyl methacrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, tridecyl methacrylate, hydroxyethyl acrylate, hydroxypropyl acrylate, acrylamide, dimethylacrylamide, acrylonitrile, dimethylaminoethyl acrylate, dimethylaminoethyl methacrylate, tert-butylaminoethyl acrylate, tert-butylaminoethyl methacrylate, methoxyethyl acrylate, methoxyethyl methacrylate, and the like. Additional examples of appropriate acrylic adhesives suitable in the practice of the invention are described in Satas, “Acrylic Adhesives,” pp. 396-456 in Satas, ed., Handbook of Pressure-Sensitive Adhesive Technology, 2nd ed., New York: Van Nostrand Reinhold (1989), which is hereby incorporated by reference. Many acrylic adhesives are commercially available, e.g., from National Starch and Chemical Corporation, Bridgewater, N.J. and Solutia, Mass. Further examples of polyacrylate-based adhesives are as follows, identified as product numbers, manufactured by National Starch (Product Bulletin, 2000): 87-4098, 87-2287, 87-4287, 87-5216, 87-2051, 87-2052, 87-2054, 87-2196, 87-9259, 87-9261, 87-2979, 87-2510, 87-2353, 87-2100, 87-2852, 87-2074, 87-2258, 87-9085, 87-9301 and 87-5298.

Suitable acrylic polymers can comprise cross-linked and non-cross-linked polymers. The polymers can be cross-linked by known methods to provide the desired polymers. For example, in certain embodiments, the adhesive is a polyacrylate adhesive having a glass transition temperature (Tg) less than −10° C., such as from about −20° C. to about −35° C. The molecular weight of the polyacrylate adhesive, expressed as weight average (“MW”), can range from about 25,000 to about 10,000,000, such as from about 50,000 to about 3,000,000 and/or from about 100,000 to about 1,000,000 prior to any cross-linking reactions. Upon cross-linking the MW approaches infinity, as known to those involved in the art of polymer chemistry.

The dosage form can include still other materials (e.g., in addition to the drug and matrix and in addition to the optional backing and adhesive layers discussed above).

Illustratively, the dosage form can further include various other components, such as osmotic agents, permeation enhancers, stabilizers, dyes, diluents, plasticizers, tackifying agents, pigments, carriers, inert fillers, antioxidants, excipients, gelling agents, anti-irritants, vasoconstrictors, anti-inflammatory materials (e.g., steroids and/or other anti-inflammatory materials which can reduce, for example, redness), and the like. These other components can be located in the aforementioned backing layer, adhesive layer, or matrix layer, of they may be located in other layers or elsewhere in the dosage form.

For example, in one embodiment of the present invention, the dosage form can include a fluid imbibing pump. “Fluid imbibing pump”, as used herein, is meant to encompasses a class of devices which deliver their contents, upon exposure to an external fluid, at a rate corresponding to the rate at which the external fluid is imbibed into the pump. These devices are known to the art and operate on diffusional and osmotic principals and are disclosed, for example, in U.S. Pat. Nos. 4,655,766, 4,435,027, 4,327,725, 4,210,139, 4,203,442, 4,203,440, 4,111,203, 4,111,202, 4,016,880, 3,995,631, 3,987,790, 3,916,899, 3,845,770, and 3,760,984, which are hereby incorporated by reference. For example, one illustrative osmotically driven fluid imbibing pump includes a semipermeable membrane that is permeable to an external fluid (e.g., water or water vapor from the skin or mucosa, water or water vapor from the environment surrounding the dosage form, such as from the gastrointestinal tract, etc.) but impermeable to a solute or other osmotic agent contained in the fluid imbibing pump and, optionally, impermeable to drug. The fluid imbibing pump is in fluid communication with the portion of the dosage form containing the matrix and drug, and an impermeable exterior wall member or other backing layer extends over the osmotic device and over the portion of the dosage form containing the matrix and drug. The exterior wall member or other backing layer is adapted to be attached, as by adhesive, to the skin or mucosal membrane to define an area of skin or mucosal membrane to which the drug is to be administered. As external fluid is imbibed through the semipermeable membrane (e.g., water or water vapor from the skin) into the fluid imbibing pump, liquid is discharged at a controlled rate from the fluid imbibing pump through an opening in the membrane or through an opening in a suitable housing structure for the fluid imbibing pump. The liquid thus discharged from the fluid imbibing pump increases the pressure in the portion of the dosage form containing the drug and matrix and thus influences migration of the drug toward the skin area or the mucosal membrane area defined by the system's external wall covering. In this manner, the semipermeable membrane can be used to control delivery rate independent of skin or mucosal membrane permeability.

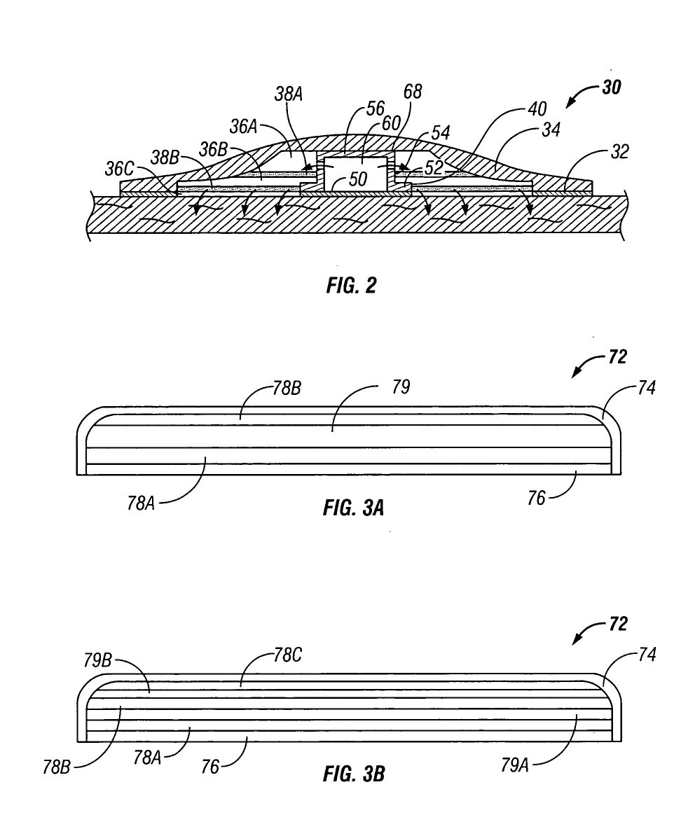

One embodiment of the present invention in which the dosage form includes a fluid imbibing pump is illustrated in FIG. 2. Referring to FIG. 2, there is shown dosage form 30 which includes circumferential adhesive layer 32, backing layer 34, a plurality of matrix layers 36a, 36b, and 36c and a plurality of drug layers 38a and 38b. Dosage form 30 further includes osmotic pump 40. Osmotic pump 40 includes a semipermeable membrane 50 extending over and firmly attached to the outer surface of outwardly directed flange 52 on the open end of a generally cylindrical cup 54 having end wall 56 and side wall 58 defining closed chamber 60. One or more outlet openings 68 are provided in side wall 58 of cup 54, and cup 54 is filled with an aqueous solution or other fluid containing an osmotic agent. In operation, when fluid is imbibed, e.g., from the subject's skin or mucosa, through semipermeable membrane 50 into chamber 60, the aqueous solution or other fluid in chamber 60 is forced out through outlet openings 68 and into the matrix and drug portion of the dosage form.

It will be appreciated that, although the embodiment illustrated in FIG. 2 shows matrix and drug as being configured as alternating discrete layers, such need not be the case, and matrix and drug can present in any of the configurations set forth in FIGS. 1A-1E.

Moreover, it will be appreciated that, although the embodiment illustrated in FIG. 2 shows one particular osmotic pump configuration, other suitable osmotic pump configurations can be employed, for example, as in the case where the closed chamber comprises two sub-chambers separated from one another by a highly flexible impermeable partition sealed to the cup's side wall or a piston that is in sliding, fluid-sealing relationship with the cup's side wall. The first sub-chamber is adjacent to the semipermeable membrane and is filled with an osmotic agent; the second sub-chamber is in fluid communication with the housing's outlets and is filled with water or a suitable aqueous solution. In operation, when fluid is imbibed, e.g., from the subject's skin or mucosa, through the semipermeable membrane into the first sub-chamber, the highly flexible impermeable partition distends into (or the piston is driven into) the second sub-chamber, compressing the second sub-chamber and forcing the water or suitable aqueous solution in the second sub-chamber through the outlet openings and into the matrix and drug portion of the dosage form.

As discussed above, the dosage form of the present invention can include still other materials (e.g., in addition to the drug and matrix and in addition to the optional backing and adhesive layers discussed above), such as one or more additional layers. For example, the above-described dosage forms can include one or more hydrophilic layers that are stratified with the drug and matrix. Illustratively, the one or more hydrophilic layers can be made from a dehydrated hydrogel, such as a cross-linked poly(acrylic acid) or a hydroxyether cellulose. As a further example, the above-described dosage forms can include one or more blocking layers having a pattern of voids (e.g., one or more holes) to control delivery of the drug to the skin or mucosa. The blocking layers can be made, for example, of a polymer which is impermeable to water, water vapor, drug, or any or all of these. Illustratively, a blocking layer containing a plurality of holes or another suitable pattern of voids can be disposed adjacent to (e.g., adjacent and proximal to) a membrane layer to regulate permeation through the membrane layer. As yet a further example, the above-described dosage forms can include a erodible layer disposed on the proximal side of a mucoadhesive layer. Illustratively, such an erodible layer can be an erodible lipid layer that is adapted to prevent adhesion of one dosage form to another, for example, when a plurality of such dosage forms are contained in an enteric or other capsule and adapted to erode quickly in the environment into which the dosage form is released after dissolution of the enteric or other capsule.

The transdermal and transmucosal dosage forms in accordance with the aspect of the present invention described above can be prepared by any suitable technique. For example, one method for preparing the above-described transdermal and transmucosal dosage forms, to which method the present invention also relates, is described in detail below.

Briefly the method includes sequentially forming two or more layers of drug and two or more layers of matrix such that the amount of drug contained in the two or more layers of drug exceeds the solubility limit of the drug in the matrix. Subsequent to the forming step or steps, the drug can become dispersed (e.g., by diffusion) in the matrix, as in the case where the drug becomes substantially uniformly dispersed in the matrix, or the drug and the matrix can remain as substantially discrete layers.

A variety of methods can be used to form the drug layers. For example, one or more of the drug layers can be formed by dispensing drops of a solution or dispersion containing the drug next to one another, for example by using a single drop dispenser or by dispensing the drops by spraying. Additionally or alternatively, one or more of the drug layers can be formed by microembossing and/or by sequential adsorption of polyelectrolytes. Matrix layers can be formed by any of the methods suitable for forming the drug layers, or they can be formed by other methods, such as those involving solvent casting, screen printing, and extrusion techniques.

Illustratively, the drug layers and the matrix layers can be sequentially formed over a substrate, such as a suitable backing layer or a pressure sensitive adhesive layer. In cases where the drug layers and the matrix layers are sequentially formed over a backing layer, the method can further include forming a pressure sensitive adhesive layer over the drug layers and the matrix layers, for example, to enclose the drug layers and the matrix layers within an enclosure formed, at least in part, by the backing layer and the pressure sensitive adhesive layer. In cases where the drug layers and the matrix layers are sequentially formed over a pressure sensitive adhesive layer, the method can further include forming a backing layer over the drug layers and the matrix layers, for example, to enclose the drug layers and the matrix layers within an enclosure formed, at least in part, by the backing layer and the pressure sensitive adhesive layer.

The present invention, in another aspect thereof, relates to a transdermal or transmucosal dosage form which includes two or more drug-containing layers and one or more intervening hydrophilic layers in which the two or more drug-containing layers are separated from one another by the one or more intervening hydrophilic layers.

Suitable drugs for use in this aspect of the present invention include those set forth above. Each of the drug-containing layers can include the same drug, or each of the drug-containing layers can contain a different drug. Still alternatively, in cases where there are more than two drug-containing layers, at least two, but fewer than all, of the drug-containing layers can contain the same drug, and the two drug-containing layers containing the same drug can be adjacent drug-containing layers, or they can be separated from one another by a drug-containing layer which contains another drug.

Each of the one or more hydrophilic layers can be made from any material which is activated slowly by water vapor, such as water vapor from the skin or from the mucosa to which the dosage form is to be applied. Illustratively, each of the one or more hydrophilic layers can be made from a dehydrated hydrogel, such as a cross-linked poly(acrylic acid) or a hydroxyether cellulose. Other examples of suitable materials from which the hydrophilic layers can be made include hydroxypropylcellulose, hydroxypropylmethylcellulose, and methyl cellulose.

Dosage forms in accordance with this aspect of the present invention can include other components (e.g., in addition to the drug-containing layers the intervening hydrophilic layers).

For example, as discussed above, the dosage form can include a backing layer. The backing layer is generally selected so as to be impermeable to the drug contained in the dosage form and so as to seal the dosage form from the external environment. The backing layer can comprise, for example, a polymer which is insoluble in and impermeable to aqueous media, such as those discussed above.

Also as discussed above, the dosage form can include a mucoadhesive or other adhesive layer. The adhesive layer can comprise an adhesive such as a pressure sensitive adhesive, suitable examples of which include those discussed above.

Still additionally or alternatively, the dosage form can include one or more blocking layers containing a pattern of voids (e.g., a plurality of holes) to control delivery of the drug to the skin or mucosa. The blocking layers can be made, for example, of a polymer which is impermeable to water, water vapor, drug, or any or all of these. For example, a blocking layer containing a plurality of holes or another suitable pattern of voids can be disposed adjacent to (e.g., adjacent and proximal to) a membrane layer to regulate permeation through the membrane layer.

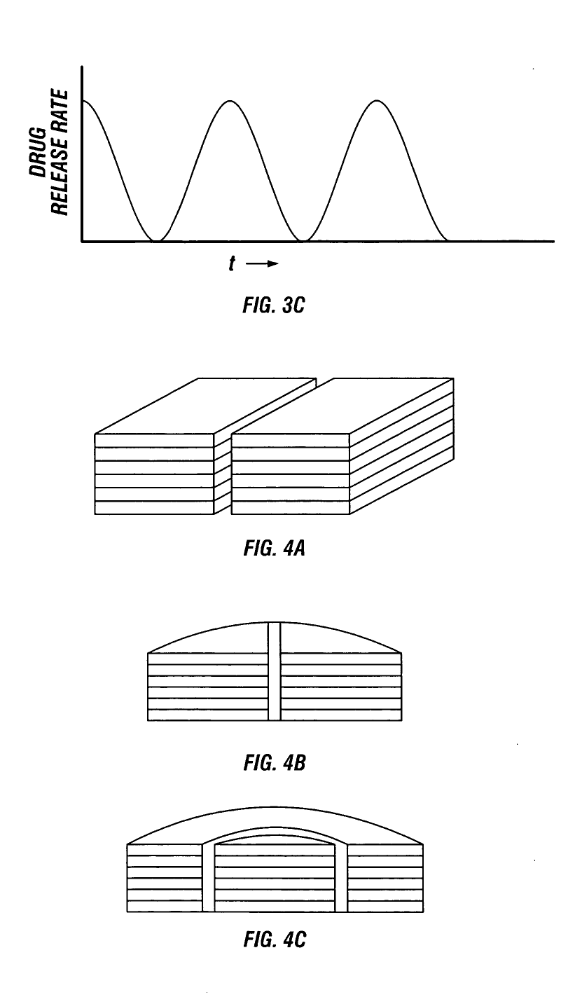

For example, suitable configurations of drug containing layers/hydrophilic layers are illustrated in FIGS. 3A-3B.

Referring to FIG. 3A, there is shown dosage form 72 which includes backing layer 74 and adhesive layer 76 which together enclose first drug layer 78a and second drug layer 78b, first drug layer 78a and second drug layer 78b being separated from one another by hydrophilic layer 79. As discussed above, the drug contained in first drug layer 78a can be the same as the one contained in second drug layer 78b, or the drug contained in first drug layer 78a can be different than the one contained in second drug layer 78b.

Referring to FIG. 3B, there is shown dosage form 72 which includes backing layer 74 and adhesive layer 76 which together enclose first drug layer 78a, second drug layer 78b, and third drug layer 78c, first drug layer 78a and second drug layer 78b being separated from one another by hydrophilic layer 79a, and second drug layer 78b and third drug layer 78c being separated from one another by hydrophilic layer 79b. As discussed above, the drug contained in each of drug layers 78a, 78b, and 78c can be the same; or the drug contained in each of drug layers 78a, 78b, and 78c can be different from one another; or the drug contained in each of drug layers 78a and 78b can be the same but different from the drug contained in drug layer 78c; or the drug contained in each of drug layers 78a and 78c can be the same but different from the drug contained in drug layer 78b. In each of the foregoing embodiments discussed in relation to FIG. 3B, hydrophilic layers 79a and 79b can be the same or different.

As discussed above, the dosage form of this aspect of the present invention, includes two or more drug-containing layers and one or more intervening hydrophilic layers. Generally, these layers are situated one above the other to form a set of layers. In one embodiment, the dosage form of this aspect of the present invention can contain only one such set of layers. In an alternative embodiment, the dosage form can include a second set of layers. Where there are two sets of layers, the sets can be separated from one another, for example, using a barrier material which is insoluble in and impermeable to aqueous media, such as those discussed above as being suitable for forming the backing layer. Alternatively, the sets can abut one another without any barrier material.

The configuration of the two sets of layers is not particularly critical. Illustratively, the two sets of layers can be disposed in a side-by-side orientation, for example, as in the case where each set has substantially rectangular layers (e.g., in cases where the overall dosage form is a square transdermal patch, as illustrated in FIG. 4A); or they can be disposed in a side-by-side orientation where each set has substantially semicircular layers (e.g., in the cases where the overall dosage form is a circular transdermal patch, as illustrated in FIG. 4B); or they can be disposed concentrically, as in the case where one set has substantially circular layers and the other set has substantially annular layers (e.g., in the cases where the overall dosage form is a circular transdermal patch, as illustrated in FIG. 4C).

The thickness of each of the layers within any set of layers can be the same or they can be different. The thickness of the drug layers within any set of layers can be the same, or they can be different. In cases where there is more than one hydrophilic layer, the thickness of the hydrophilic layers within any set of layers can be the same, or they can be different. The thickness of particular drug and hydrophilic layers are typically selected based on the desired profile of drug delivery. Illustratively, the hydrophilic layers can have thicknesses ranging from about 0.5 micron to about 100 micron, such as from about 5 micron to about 50 and/or from about 5 micron to about 100 micron; and the drug layers can have thicknesses ranging from about 100 nm to about 100 micron, such as from about 1 micron to about 10 micron. The drug layers can contain drug and only drug. Alternatively, the drug layers can contain excipients, such as those described hereinabove. The drug layers (with or without excipients) can also include a suitable matrix. Where matrices are employed, the drug can be substantially completely dissolved in the matrix, or, alternatively, the total amount of the drug present in a drug layer can exceed the solubility limit of the drug in the matrix present in that layer, for example, as described hereinabove.

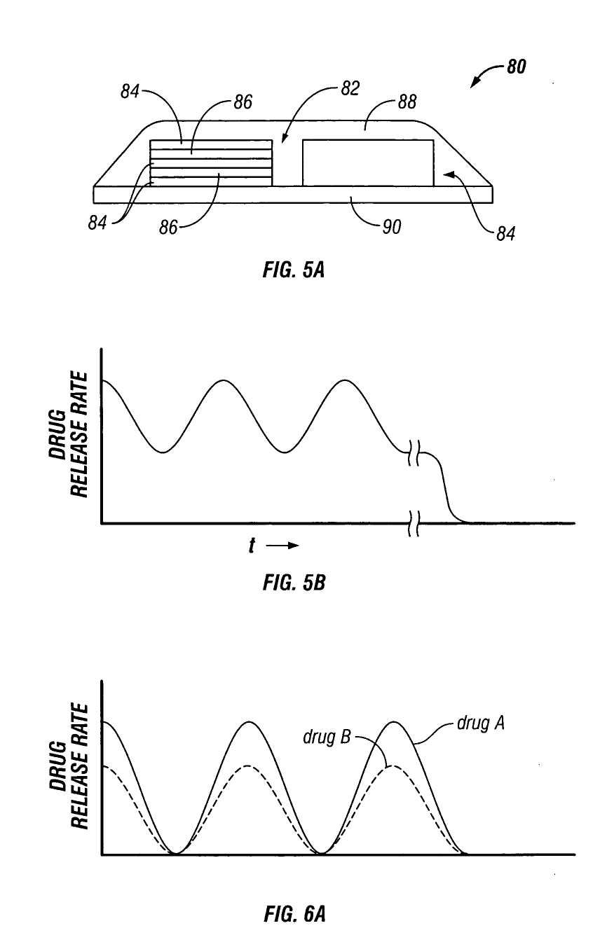

For example, where a pulsatile drug delivery profile (e.g., as shown in FIG. 3C) is desired, one can employ a dosage form having the cross-section shown in FIG. 3B. As one skilled in the art will appreciate, the duration of a particular pulse can be increased or decreased by increasing or decreasing the thickness of the relevant drug layer, the concentration of the drug in the drug layer's matrix (where a matrix is employed), the nature of the matrix in the drug layer (where a matrix is employed), the drug's water solubility, etc. The length of time between any two particular pulses can be increased or decreased by increasing or decreasing the thickness of the relevant hydrophilic layer, the rate at which the hydrophilic layer is activated from an impermeable state to a permeable state (for example, by water vapor from the skin), etc. For a pulsatile drug delivery profile having a regular period, one can employ a dosage form having the cross-section shown in FIG. 4A in which each drug layer has substantially the same thickness as every other drug layer and in which each hydrophilic layer has substantially the same thickness as every other hydrophilic layer.

Where a more complex drug release pattern is desired, for example, a pulsatile drug delivery profile superimposed on a steady-state delivery profile, one can employ a dosage form having the cross-section shown in FIG. 5A. Referring to FIG. 5A, dosage form 80 includes layer set 82 and constant release portion 84, each of which contain the same drug X. In layer set 82, drug-containing layers 84 are separated from one another by intervening hydrophilic layers 86. Constant release portion 84 can be formulated using conventional techniques (for example, by dissolving drug X in a suitable matrix), or constant release portion 84 can include a matrix and drug-X wherein the total amount of drug X present in constant release portion 84 exceeds the solubility limit of drug X in the matrix, for example, as described hereinabove. Dosage form 80 can further include backing layer 88 and adhesive layer 90 and can produce a drug release profile as shown in FIG. 5B.

The dosage forms of this aspect of the present invention can be used to deliver more than one drug.

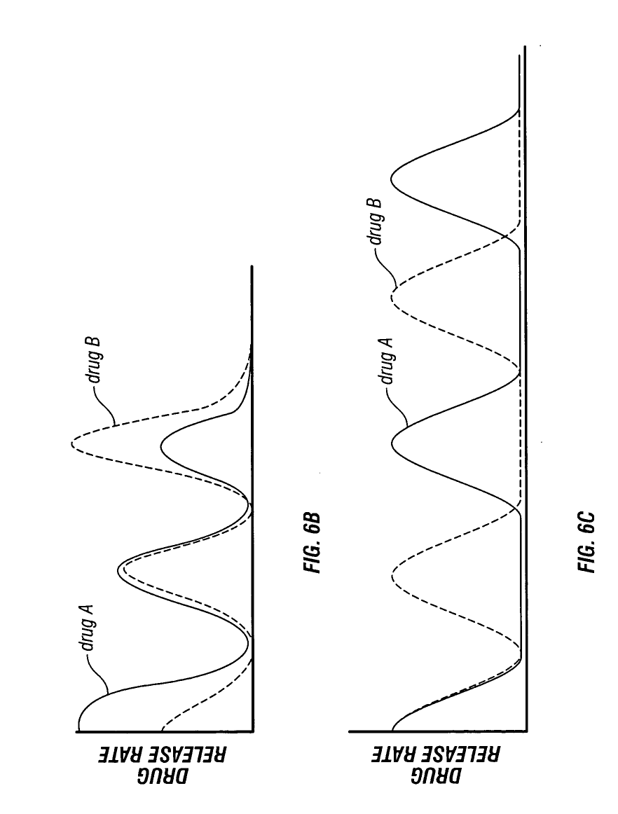

For example, drug A and drug B can be delivered together in a pulsatile manner by using the dosage form illustrated in FIG. 3B and by incorporating drug A and drug B in each of first drug layer 78a, second drug layer 78b, and third drug layer 78c. A typical drug release profile produced by such a configuration is shown in FIG. 6A.

By varying the amounts and/or concentrations of drug A and drug B in the various drug layers (e.g., referring to the embodiment shown in FIG. 3B, in first drug layer 78a, second drug layer 78b, and third drug layer 78c), one can readily alter the drug release profile of the two drugs with respect to one another, for example, to produce a drug release profile like the one illustrated in FIG. 6B.

Still alternatively, by having drug A present only in alternate drug layers and drug B present only in the remaining drug layers, a drug release profile like the one illustrated in FIG. 6C can be produced.

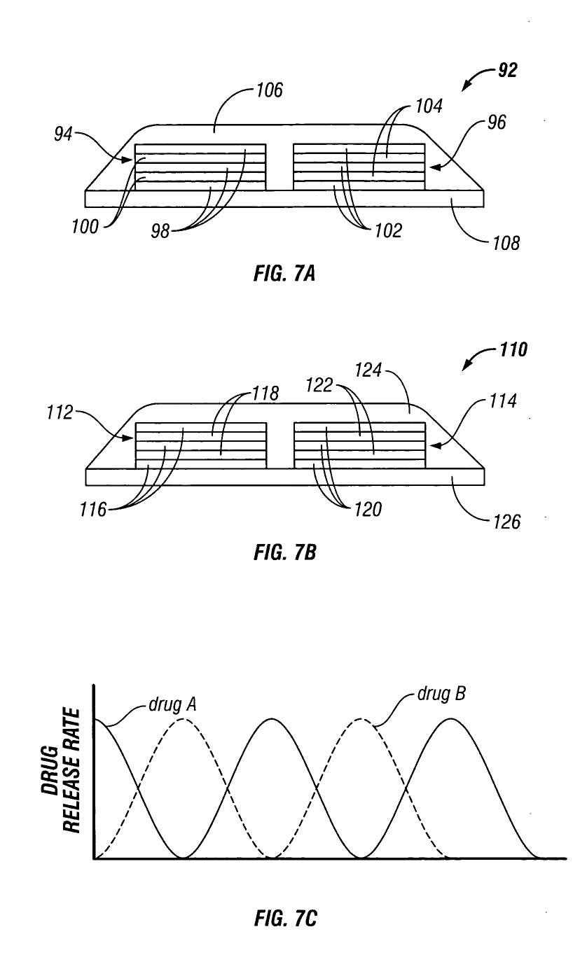

The drug release profiles illustrated in FIGS. 6A, 6B, and 6C can also be produced using dosage forms having a plurality of layer sets. For example, the drug release profile illustrated in FIGS. 6A and 6B can be produced using a dosage form having two sets of layers in which each of the first layer set's drug-containing layers is substantially aligned in a coplanar arrangement with each of the second layer set's drug-containing layers and in which each of the first layer set's hydrophilic layers is substantially aligned in a coplanar arrangement with each of the second layer set's hydrophilic layers. Illustratively, one such dosage form is shown in FIG. 7A. Referring now to FIG. 7A, dosage form 92 includes layer set 94 and layer set 96. In layer set 94, drug-containing layers 98 contain drug A and are separated from one another by intervening hydrophilic layers 100. In layer set 96, drug-containing layers 102 contain drug B and are separated from one another by intervening hydrophilic layers 104. Dosage form 92 can further include backing layer 106 and adhesive layer 108.

Dosage forms having two or more layer sets can be used to deliver two or more drugs with independent pulsatile drug delivery profiles. For example, drug A and drug B can be delivered with pulsatile drug delivery profiles that are 180 degrees out of phase with one another (where drug A attains maximum release rate substantially at the same time that drug B's release rate is at a minimum and where drug B attains maximum release rate substantially at the same time that drug A's release rate is at a minimum). For example, such a drug release profile can be produced using a dosage form having two sets of layers in which each of the first layer set's drug-containing layers is substantially aligned in a coplanar arrangement with each of the second layer set's hydrophilic layers and in which each of the first layer set's hydrophilic layers is substantially aligned in a coplanar arrangement with each of the second layer set's drug-containing layers. Illustratively, one such dosage form is shown in FIG. 7B. Referring now to FIG. 7B, dosage form 110 includes layer set 112 and layer set 114. In layer set 112, there are five layers: three drug-containing layers 116 which contain drug A and two hydrophilic layers 118 which separate drug-containing layers 116 from one another. In layer set 114, there are five layers: three hydrophilic layers 120 and two drug-containing layers 122 which contain drug B and which are separated from one another by the middle hydrophilic layer. Dosage form 110 can further include backing layer 124 and adhesive layer 126 and can produce a drug release profile as shown in FIG. 7C.

It will be appreciated that drug A and drug B can be delivered with pulsatile drug delivery profiles that are phase-shifted to any desired degree, for example, by adjusting the thickness of the first hydrophilic layer in the layer set whose first layer (i.e., the layer intended to be closest to the skin) is not a drug-containing layer. For example, by using layer sets in which all hydrophilic layers are the same thickness except the first hydrophilic layer in the layer set whose first layer (i.e., the layer intended to be closest to the skin) is not a drug-containing layer, one can produce a pulsatile drug delivery profile where each of drug A and drug B are delivered at regular intervals (e.g., with a period, T, of from about 1 hour to about 3 days) and where the maximum of drug A and the maximum of drug B are temporally offset by, for example, about 0.05 T, about 0.1 T, about 0.15 T, about 0.2 T, about 0.25 T about 0.3 T, about 0.35 T, about 0.4 T, about 0.45 T, or about 0.5 T, the last of these being achieved when all hydrophilic layers (including the first hydrophilic layer in the layer set whose first layer (i.e., the layer intended to be closest to the skin) is not a drug-containing layer) are the same thickness.

It will be further appreciated that drug delivery profiles can be modified by using a hydrophilic layer having a hole, a plurality of holes, a suitable pattern of voids, a suitable pattern of thickness variation, etc.

The present invention, in yet another aspect thereof, relates to a method for delaying release of an active from an active layer disposed in a dosage form which includes, in addition to the active layer, an adhesive layer. The method involves disposing one or more hydrophilic layers between the adhesive layer and the active layer. Suitable hydrophilic layers for use in connection with this aspect of the present invention include those containing dehydrated hydrogel, such as a cross-linked poly(acrylic acid) or a hydroxyether cellulose. “Active”, as used in this context, is meant to include drugs, such as those set forth hereinabove. Suitable adhesive layers for use in connection with this aspect of the present invention include, for example, those containing pressure sensitive adhesives, such as the ones described hereinabove.

As indicated above, the method involves disposing one or more hydrophilic layers between the adhesive layer and the active layer. For the purposes of this aspect of the present invention, a hydrophilic layer is to be deemed to be between an active layer and an adhesive layer if, when traveling from the plane in which the adhesive layer lies to the active layer, one crosses the hydrophilic layer.

For example, the adhesive layer can be a continuous layer which forms one face of the dosage form, the hydrophilic layer can be disposed adjacent to the adhesive layer, and the active layer can be disposed adjacent to the hydrophilic layer. Alternatively, the adhesive layer can be a continuous layer which forms one face of the dosage form, the hydrophilic layer can be disposed adjacent to the adhesive layer, and the active layer can be disposed in the dosage form but not adjacent to the hydrophilic layer (for example, as in the case where there exists one or more intervening layers between the active layer and the hydrophilic layer). Still alternatively, the adhesive layer can be a continuous layer which forms one face of the dosage form, the hydrophilic layer can be disposed in the dosage form but not adjacent to the adhesive layer (for example, as in the case where there exists one or more intervening layers between the adhesive layer and the hydrophilic layer), and the active layer can be disposed adjacent to the hydrophilic layer and on the side of the hydrophilic layer which is remote from the adhesive layer. Still alternatively, the adhesive layer can be a continuous layer which forms one face of the dosage form, the hydrophilic layer can be disposed in the dosage form but not adjacent to the adhesive layer (for example, as in the case where there exists one or more intervening layers between the adhesive layer and the hydrophilic layer), and the active layer can be disposed in the dosage form on the side of the hydrophilic layer which is remote from the adhesive layer but not adjacent to the hydrophilic layer.

As one skilled in the art will appreciate, the adhesive layer does not need to be a continuous layer which forms one face of the dosage form. For example, the adhesive layer is frequently disposed only around the perimeter of the dosage form. In any event, the adhesive layer, whatever the pattern it forms on the face of the dosage form, typically lies in a plane.

Accordingly, in other embodiments of this aspect of the present invention, the adhesive layer can be present as continuous or intermittent ribbon around the perimeter of the dosage form, the hydrophilic layer can be disposed adjacent to the plane defined by this ribbon, and the active layer can be disposed adjacent to the hydrophilic layer. Alternatively, the adhesive layer can be present as continuous or intermittent ribbon around the perimeter of the dosage form, the hydrophilic layer can be disposed adjacent to the plane defined by this ribbon, and the active layer can be disposed in the dosage form but not adjacent to the hydrophilic layer (for example, as in the case where there exists one or more intervening layers between the active layer and the hydrophilic layer).

The present invention, in yet another aspect thereof, relates to a method for delaying delivery of an active from an active layer disposed in a dosage form to a subject's skin or mucosa. The method includes disposing, in the dosage form, one or more hydrophilic layers between the active layer and the subject's skin. In this embodiment, the hydrophilic layer can form the outermost layer of the dosage form (e.g., as in the case where, in use, the dosage form is affixed to the subject's skin with the hydrophilic layer contacting or proximal to the subject's skin). Alternatively, the outermost layer of the dosage form can be a layer other than the hydrophilic layer (e.g., an adhesive layer or some other layer) with the hydrophilic layer being distal to the dosage form's outermost layer and with the active layer being distal to the hydrophilic layer.

The dosage forms and methods described hereinabove (e.g., dosage forms which include a matrix and a drug in which the total amount of drug present in the dosage form exceeds the solubility limit of the drug in the matrix; methods for making such dosage forms; dosage forms which include two or more drug-containing layers and one or more intervening hydrophilic layers in which the two or more drug-containing layers are separated from one another by the one or more intervening hydrophilic layers; and methods for delaying release of an active from an active layer disposed in a dosage form) can be practiced using any suitable fabrication techniques for preparing transdermal and transmucosal patches and other transdermal and transmucosal dosage forms having layered configurations. For example, the dosage forms described hereinabove can be fabricated using conventional techniques, such as those involving traditional solvent casting methods, extrusion methods, and lamination methods. The dosage forms described hereinabove can also be produced using the methods described hereinbelow, to which the present invention also relates.

The present invention also relates to a method of manufacturing a transdermal or transmucosal dosage form. The method includes providing a substrate and disposing at least one transdermal or transmucosal dosage form layer on the substrate using a printing process.

“Substrate”, as used in this context, is meant to include any substantially planar material. Illustratively, the substrate can be part of the dosage form, for example, as in the case where the substrate is a backing layer of the dosage form, an adhesive layer of the dosage form, or a release liner of the dosage form. Alternatively, the substrate can be a planar material which is not part of the dosage form, such as in the case where the substrate functions only as a convenient surface on which to build the dosage form or parts of the dosage form. The substrate can be made of a single layer or it can include plurality of layers, which layer or layers can be formed by any suitable method, such as by solvent casting methods, extrusion methods, or printing methods described herein.

As indicated above, the method involves disposing at least one transdermal or transmucosal dosage form layer on the substrate using a printing process. The layer disposed in this manner can be, for example, a backing layer, an adhesive layer, an osmotic membrane layer, a blocking layer, a drug layer, a hydrophilic layer, a barrier layer, a structural matrix or other matrix layer, or any other layer of a transdermal or transmucosal dosage form. Illustratively, the substrate can be a release liner, and the method can be carried out by disposing an adhesive layer on the release liner.

The layer can be uniform in composition or it can be patterned, for example, as in the case where one portion of the layer contains a first drug and another portion of the layer contains a second drug; as in the case where one portion of the layer contains a first drug and another portion of the layer contains a hydrophilic polymer; as in the case where the layer is a blocking layer containing a pattern of voids (e.g., a plurality of holes) to control delivery of the drug to the skin or mucosa or to regulate permeation through an adjacent membrane layer; or as in the case where the layer is a hydrophilic layer containing a pattern of voids (e.g., a plurality of holes), for example, to modify the drug delivery profile.

“Printing process”, as used herein means a process which involves one or more of the following: (i) single drop dispensers; (ii) multiple drop dispensers; (iii) microembossing; and (iv) sequential adsorption of polyelectrolytes.

Single drop dispensers are those which eject single drops of fluid, such as in the form of a dispersion, in the form of a solution, or in liquid form, with a controlled size. Suitable drop sizes can range from about 5 pL to 1 nL, such as from about 50 pL to about 250 pL. Single drop dispensers are meant to include drop-on-demand dispensers as well as continuous inkjet and micropipette dispensers. Examples of drop-on-demand dispensers include thermal drop-on-demand dispensers, piezoelectric drop-on-demand dispensers, electrostatic drop-on-demand dispensers, acoustic drop-on-demand dispensers, as well as drop-on-demand dispensers controlled by microelectromechanical systems (“MEMS”).

Illustratively, suitable single drop dispensers which can be used in the practice of the method of the present invention include ink jet printers which utilize piezoelectric dispensers to dispense liquid drops in rapid succession (e.g., at rates of up to at least 2,000 drops per second).

In one such system (known as a continuous device), a fluid under pressure issues from an orifice in a dispenser while a piezoelectric crystal attached to the dispenser induces pressure oscillations in the fluid causing the fluid stream to break into drops after issuing from the dispenser. The drops form in the presence of an electrostatic field and thus acquire an electric charge. As the drops continue toward the substrate, they pass through another electrostatic field which interacts with their acquired charge to deflect them to a desired location.

In another such system (known as a drop-on-demand device), fluid from a reservoir is fed into a dispenser and a piezoelectric crystal directly or indirectly coupled to the fluid responds to a voltage pulse to induce a volume change in the dispenser, thus causing a drop of fluid to issue from an orifice toward a substrate. In this type of dispenser a drop is formed only in response to a predetermined voltage pulse.

Other suitable single drop dispensers include ink jet systems that use heat to form and propel drops of fluid. Thermal ink jets heat a fluid so rapidly that the fluid vaporizes. Rapid volumetric changes provide the force for propelling drops of fluid from the dispenser.

These and other single drop dispensers suitable for use in the practice of the method of the present invention are described, for example, in U.S. Pat. Nos. 4,313,684, 4,339,762, 4,490,728, 4,514,741, 4,683,481, 4,812,859, 4,870,433, 4,877,745, 4,887,098, 5,278,584, 5,338,688, 5,474,796, 5,449,754, 5,658,802, 5,700,637, 5,734,399, 5,793,393, 6,270,201, 6,491,377, 6,592,197; in various articles in the Hewlett-Packard Journal, Vol. 36, No. 5 (May 1985), Vol. 39, No. 4 (August 1988), Vol. 39, No. 5 (October 1988), Vol. 43, No. 4 (August 1992), Vol. 43, No. 6 (December 1992) and Vol. 45, No. 1 (February 1994) editions; and in Lloyd et al., chapter 13 in Durbeck et al., eds, Output Hardcopy Devices, San Diego: Academic Press (1988); which are hereby incorporated by reference.

Irrespective of the particular type of single drop dispenser being used, the materials to be printed (e.g., polymer, drug, excipients, osmotic agents, etc.) are solubilized or dispersed in a liquid having a viscosity and/or surface tension that is compatible with the single drop dispenser being employed. For example, for single drop inkjet dispensing, the solution or dispersion can have a viscosity of from about 2 cP to about 10 cP and a surface tension of from about 25 dynes/cm to about 35 dynes/cm. Where polymer solutions are being dispensed, relatively low concentrations (such as less than about 1 percent by weight) may be desirable to keep viscosities low. Additionally or alternatively, viscosities can be controlled by modulating the temperature of the solution and/or by the use of surfactants. The choice of solvent is not particularly critical. Illustratively, mucoadhesive polymers, such as carbomer, polycarbophil, sodium carboxy methyl cellulose, and chitosan, can be printed from an aqueous or alcoholic solution. Structural or matrix polymers, such as mixed or substituted hydroxyalkyl ethers of cellulose, cellulose esters, polyisobutylene, polybutadiene, and ethylenevinyl acetate copolymer, can be printed from aqueous or organic solvents.

To print a layer using a single drop dispenser, a plurality of drops are dispensed onto the substrate next to each other. This can be carried out using an array of inkjet nozzles, by translating a single inkjet nozzle by a defined distance, or by translating the substrate, or by any combination of these techniques. The area that a single drop covers influences, in part, the optimal center-to-center drop distance. This area, in turn, depends, at least in part, on the rate at which the drops dry. The rate at which the drops dry can be controlled, for example, by choice of solvent and/or by the temperature during drop impact with the substrate. The rate at which the drops dry, in addition to influencing the optimal center-to-center drop distance, also influences the ability of neighboring drops to merge together and the degree to which drops subsequently dispensed on top can mix. For example, to obtain discrete layers in which there is minimal mixing or blending between the layers, the solvent used in one layer can be chosen so as not to be a solvent for the other layer. Additionally or alternatively, to obtain discrete layers, the drying kinetics of the drops can be manipulated (for example, by heating with convention or infrared radiation) so as to be sufficiently fast that resolubilization of the previous layer is minimized. Still additionally or alternatively, to obtain discrete layers, printing the next layer can be delayed until the previous layer is completely dry.

The thickness of a given layer depends, in part, on drop volumes, the concentrations of the dispensing solutions, surface tension, viscosity, drying kinetics, and/or temperature. Using low concentrations for the dispensing solutions (i.e., less than about 1 percent by weight) and small drop volumes (e.g., from about 5 pL to about 250 pL), layer thicknesses of from about 10 nm to about 100 nm can be achieved. Where a thicker layer is desired, for example, where a mucoadhesive layer having a thickness of from about 100 nm to about 1 micron is desired, such a thicker layer can be achieved by overprinting.

It will be appreciated that not all layers need be disposed using single drop dispensers. For example, one or some of the layers can be disposed using single drop dispensers, while others can be formed using other printing processes (e.g., using multiple drop dispensers, using microembossing, and/or using sequential adsorption of polyelectrolytes), and still others can be disposed using traditional techniques, such as solvent casting, extrusion, and lamination techniques.

As briefly discussed above, the method of this aspect of the present invention can involve disposing at least one layer on the substrate using a multiple drop dispenser printing process. Multiple drop dispensers are meant to include those which involve spraying devices that operate by air-assisted atomization, ultrasonic-assisted atomization, and piezoelectric-assisted atomization.

Irrespective of the particular type of multiple drop dispenser being used, the materials to be printed (e.g., polymer, drug, excipients, osmotic agents, etc.) are solubilized or dispersed in a liquid having a viscosity and/or surface tension that is compatible with the multiple drop dispenser being employed. For example, the solution or dispersion can have a viscosity of from about 2 cP to about 10 cP and a surface tension of from about 25 dynes/cm to about 35 dynes/cm. Where polymer solutions are being dispensed, relatively low concentrations (such as less than about 1 percent by weight) may be desirable to keep viscosities low. Additionally or alternatively, viscosities can be controlled by modulating the temperature of the solution and/or by the use of surfactants. The choice of solvent is not particularly critical. Illustratively, mucoadhesive polymers, such as carbomer, polycarbophil, sodium carboxy methyl cellulose, and chitosan, can be printed from an aqueous or alcoholic solution. Structural or matrix polymers, such as mixed or substituted hydroxyalkyl ethers of cellulose, cellulose esters, polyisobutylene, polybutadiene, and ethylenevinyl acetate copolymer, can be printed from aqueous or organic solvents.

When using multiple drop dispensers, each layer thickness is influenced by the volumetric dispensing rate, the spray area, and the concentration of the solution. As with single drop dispensers, discreteness of adjacent layers printed by multiple drop dispensers depends on solvent compatibility and drying kinetics.

Also as briefly discussed above, the method of this aspect of the present invention can involve disposing at least one layer on the substrate using microembossing techniques. In microembossing, a stamp is made from an elastomeric polymer, such as polydimethylsiloxane, or other suitable polymer. The stamp is then contacted with a solution or dispersion under conditions effective to fill the interstices of the stamp. The stamp, with its interstices filled with the solution or dispersion, is then contacted with the substrate. Alternatively, a thin layer of solution or dispersion, thicker than the tallest feature of the stamp, is spread on the substrate, and the stamp is pressed into the thin layer. In either case, upon drying, the embossed material remains in the area of the stamp's interstices.

Also as briefly discussed above, the method of this aspect of the present invention can be carried out by disposing at least one layer on the substrate using techniques involving the sequential adsorption of polyelectrolytes. In one embodiment of this technique, alternating positively charged polymers and negatively charged polymers are adsorbed from dilute aqueous solutions (i.e., from solutions in which the polymer is present below its solubility limit) onto a substrate. In another embodiment of this technique, alternating hydrogen-donating polymers and hydrogen-accepting polymers are adsorbed from dilute aqueous solutions (i.e., from solutions in which the polymer is present below its solubility limit) onto a substrate. In either embodiment, the substrate can be charged or uncharged. In some applications, an initial priming polymer, such as a polymer that is highly charged in a number of states and that has a significant hydrophobic backbone (e.g., branched polyethyleneimine) can be adsorbed first to the substrate to enhance adhesion with the substrate. Each adsorbed layer thickness typically ranges from about 1 nm to about 10 nm, depending on the solution's ionic strength and/or pH and depending on the polymer's molecular weight.

The following examples further illustrate the present invention.

### EXAMPLES

### Example 1

### Fabrication of a Gastrointestinal Patch Using a Single Drop Dispenser

This example describes a method for making a gastrointestinal (“GI”) patch with an osmotic push-pull engine. The patch will have a low profile and will fit in a size 00 capsule. The diameter of the disk will be 4 mm or smaller, and the aspect ratio will be 1 (total disk thickness) to 10 (disk diameter) or smaller. The disk will consist of 6 strata: mucoadhesive, membrane, drug, membrane, osmotic agent, and membrane. In general, the fabrication procedure is to sequentially print multiple layers of each material to constitute a stratum with drying between successive printed layers and between successive strata.

A TEFLON coated surface (or other surface having chemistry which permits easy release of the printed patch) is used.

First, carbomer (or another mucoadhesive polymer) is printed by inkjet from an aqueous or mixed aqueous-alcoholic solution onto the TEFLON coated surface in a suitable pattern, such as an annular ring or a filled circle. Since drying of the mucoadhesive layer may be slow because of the slow evaporation of water and the hydrophobicity of the TEFLON surface, heating (e.g., by convention or by infrared heating) can be used to speed up the drying process. Once the first mucoadhesive layer is dry, it can be overprinted with the same mucoadhesive solution or with a different mucoadhesive solution. The process is repeated until the mucoadhesive layer has achieved the desired thickness (e.g., between about 100 nm and about 10 micron, such as between about 100 nm and about 1 micron). The mucoadhesive layer is then allowed to completely dry prior to printing the next layer.

The first membrane layer is then printed over the dried mucoadhesive layer by inkjet from a solution of a membrane polymer (e.g., cellulose acetate or ethyl cellulose) in an organic solvent (e.g., acetone). The shape of the membrane layer is that of a filled circle having a diameter slightly greater than the diameter of the mucoadhesive layer and having one or more unprinted holes. The membrane layer is allowed to dry. The membrane layer is then overprinted, the overprinted membrane layer is allowed to dry, and the process is repeated until the first membrane layer has achieved the desired thickness. The first membrane layer is then allowed to completely dry prior to printing the next layer.

The drug layer is then printed over the dried first membrane layer by inkjet from an aqueous solution of drug. The drug layer is printed over an area smaller than that of the underlying first membrane layer. The drug layer is allowed to dry (with optional heating, for example by convention or by infrared radiation). The drug layer is then overprinted, the overprinted drug layer is allowed to dry, and the process is repeated until the drug layer has achieved the desired thickness. The drug layer is then allowed to completely dry prior to printing the next layer.

The second membrane layer is then printed over the dried drug layer by inkjet from a solution of a membrane polymer (which can be the same membrane polymer used in the first membrane layer or a different membrane polymer) in an organic solvent (e.g., acetone). The shape of the membrane layer is that of a filled circle having a diameter substantially equal to the diameter of the first membrane layer. The membrane layer is allowed to dry. The membrane layer is then overprinted, the overprinted membrane layer is allowed to dry, and the process is repeated until the second membrane layer has achieved the desired thickness. The second membrane layer is then allowed to completely dry prior to printing the next layer.

The osmotic agent layer is then printed over the dried second membrane layer by inkjet from an aqueous or alcoholic solution of osmotic agent (e.g., polyethylene oxide). The osmotic agent layer is printed over an area smaller than that of the underlying second membrane layer. The osmotic agent layer is allowed to dry (with optional heating, for example by convention or by infrared radiation). The osmotic agent layer is then overprinted, the overprinted osmotic agent layer is allowed to dry, and the process is repeated until the osmotic agent layer has achieved the desired thickness. The osmotic agent layer is then allowed to completely dry prior to printing the next layer.

The third membrane layer is then printed over the dried drug layer by inkjet from a solution of a membrane polymer (which can be the same membrane polymer used in the first and/or second membrane layers or a different membrane polymer) in an organic solvent (e.g., acetone). The shape of the membrane layer is that of a filled circle having a diameter substantially equal to the diameter of the first and second membrane layers. The membrane layer is allowed to dry. The membrane layer is then overprinted, the overprinted membrane layer is allowed to dry, and the process is repeated until the third membrane layer has achieved the desired thickness. The third membrane layer is then allowed to completely dry.

An optional backing layer can be disposed over this structure, for example, to ensure that the drug is directed toward the mucosa to which the adhesive layer adheres. Where an optional backing layer is employed, it can be disposed over the third membrane layer by inkjet printing, or the backing layer can be formed separately (e.g., by solvent casting or extrusion techniques) and laminated to the structure.

### Example 2

### Fabrication of a Transdermal Patch Using a Single Drop Dispenser

A pressure-sensitive adhesive, such as polyisobutylene or silicone, is cast onto a peelable liner, such as silicone-coated polyester, using a conventional solvent casting process.

A series of drug and matrix layers are alternatingly printed over a portion of the solvent-cast pressure-sensitive adhesive layer.

More particularly, a matrix layer, such as a ethylene-vinyl acetate copolymer layer, a poly(vinyl alcohol) layer, or a poly(vinyl pyrrolidone) layer, is printed from an organic or alcoholic solvent (with overprinting and drying as needed to achieve the desired thickness). After final drying of the matrix layer, a drug layer is printed on top of the matrix layer from an organic or alcoholic solvent (with overprinting and drying as needed to achieve the desired thickness). After final drying of the drug layer, a second matrix layer, is printed from an organic or alcoholic solvent (with overprinting and drying as needed to achieve the desired thickness). After final drying of the second matrix layer, a second drug layer is printed on top of the second matrix layer (again with overprinting and drying as needed to achieve the desired thickness). The process of printing matrix layer followed by drug layer is repeated any number of further times until the dosage form contains the desired amount of drug.

By alternatingly printing matrix layers and drug layers, uniform dispersion of the drug at high concentrations can be achieved. The concentration of the drug can be controlled by the thickness of the drug and matrix layers. For example, typically in traditional matrix systems, the drug loading is less than 10% by weight at the solubility limit of the drug in the matrix. In the dosage forms described herein, drug loading is higher than the solubility limit of the drug in the matrix. It is believed that the excess drug (i.e., the amount over the solubility limit of the drug in the matrix) is present as a solid and does not contribute to the drug remaining in the dosage form after use. Moreover, it is believed that the excess drug acts as a constant reservoir such that, as drug leaves migrates from the dosage form into the subject's skin or mucosa, thermodynamic forces cause solid drug (e.g., from a discrete drug layer) to dissolve into the surrounding matrix, thus maintaining the concentration of the drug dissolved in the matrix at a constant level (e.g., equal to the solubility limit of the drug in the matrix). Illustratively, given the same amount of drug delivered, the same delivery characteristics, and a solubility limit of 10 wt %, the amount of drug remaining in a dosage form after use will be 25% of the initial drug loading when the dosage form is prepared with an initial drug loading of 20 wt %, as compared with 50% of the initial drug loading when the dosage form is prepared with an initial drug loading of 10 wt % (i.e., at the solubility limit).

Where pulsatile drug delivery is desired, dehydrated hydrogels, such as hydroxyalkyl ethers of cellulose or cross-linked poly(acrylic acid) can be stratified with the drug and matrix. For example, after printing the desired number of layers of drug and matrix to be delivered in one pulse, a dehydrated hydrogel layer is printed from an alcoholic solution and dried. Additional layers of drug and matrix are then printed, optionally followed by printing of a second dehydrated hydrogel layer and printing of additional layers of drug and matrix. The process is repeated a number of times depending on the number of pulses desired. The dosage form is then optionally capped by traditional lamination with an impermeable sealing layer or other backing layer, like polyethylene or a polyester.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention. Further aspects of the present invention are set forth below.

1. A transdermal or transmucosal dosage form comprising:
a matrix; and
a drug; wherein the total amount of said drug present in said dosage form exceeds the solubility limit of said drug in said matrix.

2. A dosage form according to aspect 1, wherein said drug is dispersed in said matrix at a concentration greater than the solubility limit of the drug in the matrix.

3. A dosage form according to aspect 2, wherein said drug is substantially uniformly dispersed in said matrix.

4. A dosage form according to aspect 1, wherein said drug and said matrix are present as substantially discrete layers.

5. A dosage form according to aspect 1, wherein said dosage form further comprises a pressure sensitive adhesive layer on one surface of said dosage form.

6. A dosage form according to aspect 1, wherein said dosage form further comprises a backing layer on one surface of said dosage form.

7. A dosage form according to aspect 1, wherein said dosage form further comprises a pressure sensitive adhesive layer on one surface of said dosage form and a backing layer on an opposing surface of said dosage form.

8. A dosage form according to aspect 7, wherein said pressure sensitive adhesive layer comprises a mucoadhesive.

9. A dosage form according to aspect 1, wherein said dosage form further comprises a pressure sensitive adhesive layer on one surface of said dosage form and a release liner in contact with said pressure sensitive adhesive layer.

10. A dosage form according to aspect 1, wherein said dosage form further comprises one or more hydrophilic layers stratified with said drug and matrix.

11. A dosage form according to aspect 10, wherein said one or more hydrophilic layers comprise a dehydrated hydrogel.

12. A dosage form according to aspect 11, wherein the dehydrated hydrogel is a cross-linked poly(acrylic acid).

13. A dosage form according to aspect 11, wherein the dehydrated hydrogel is a hydroxyether cellulose.

14. A transdermal or transmucosal dosage form comprising:
two or more drug-containing layers; and
one or more intervening hydrophilic layers; wherein said two or more drug-containing layers are separated from one another by said one or more intervening hydrophilic layers.

15. A dosage form according to aspect 14, wherein said one or more intervening hydrophilic layers comprises a dehydrated hydrogel.

16. A dosage form according to aspect 15, wherein said dehydrated hydrogel is a cross-linked poly(acrylic acid).

17. A dosage form according to aspect 15, wherein said dehydrated hydrogel is a hydroxyether cellulose.

18. A dosage form according to aspect 14, wherein said dosage form further comprises a pressure sensitive adhesive layer on one surface of said dosage form.

19. A dosage form according to aspect 14, wherein said dosage form further comprises a backing layer on one surface of said dosage form.

20. A dosage form according to aspect 14, wherein said dosage form further comprises a pressure sensitive adhesive layer on one surface of said dosage form and a backing layer on an opposing surface of said dosage form.

21. A dosage form according to aspect 20, wherein said pressure sensitive adhesive layer comprises a mucoadhesive.

22. A dosage form according to aspect 14, wherein said two or more drug-containing layers and said one or more intervening hydrophilic layers form a first layer set; wherein said dosage form comprises a second layer set; wherein said second layer set comprises two or more drug-containing layers and one or more intervening hydrophilic layers; and wherein said second layer set's two or more drug-containing layers are separated from one another by said second layer set's one or more intervening hydrophilic layers.

23. A dosage form according to aspect 22, wherein each of said first layer set's drug-containing layers is substantially aligned in a coplanar arrangement with each of said second layer set's drug-containing layers and wherein each of said first layer set's hydrophilic layers is substantially aligned in a coplanar arrangement with each of said second layer set's hydrophilic layers.

24. A dosage form according to aspect 22, wherein each of said first layer set's drug-containing layers is substantially aligned in a coplanar arrangement with each of said second layer set's hydrophilic layers and wherein each of said first layer set's hydrophilic layers is substantially aligned in a coplanar arrangement with each of said second layer set's drug-containing layers.

25. A method for preparing a transdermal or transmucosal dosage form comprising a matrix and a drug dispersed in the matrix, wherein the total amount of the drug present in the dosage form exceeds the solubility limit of the drug in the matrix, said method comprising:
sequentially forming two or more layers of drug and two or more layers of matrix such that the amount of drug contained in the two or more layers of drug exceeds the solubility limit of the drug in the matrix.

26. A method according to aspect 25, wherein, subsequent to said forming, the drug becomes dispersed in the matrix.

27. A method according to aspect 25, wherein, subsequent to said forming, the drug becomes substantially uniformly dispersed in the matrix.

28. A method according to aspect 25, wherein, subsequent to said forming, said drug and said matrix remain as substantially discrete layers.

29. A method according to aspect 25, wherein at least one drug layer is formed by dispensing drops of a solution or dispersion containing the drug next to one another.

30. A method according to aspect 29, wherein the drops are dispensed using a single drop dispenser.

31. A method according to aspect 29, wherein the drops are dispensed by spraying.

32. A method according to aspect 25, wherein at least one drug layer is formed by microembossing.

33. A method according to aspect 25, wherein the drug layers and the matrix layers are sequentially formed over a substrate.

34. A method according to aspect 33, wherein the substrate is a backing layer.

35. A method according to aspect 34, wherein said method further comprises:
forming a pressure sensitive adhesive layer over the drug layers and the matrix layers.

36. A method according to aspect 33, wherein the substrate is a pressure sensitive adhesive layer.

37. A method according to aspect 36, wherein said method further comprises:
forming a backing layer over the drug layers and the matrix layers.

38. A method for delaying release of an active from an active layer disposed in a transdermal or transmucosal dosage form comprising, in addition to the active layer, an adhesive layer, said method comprising:
disposing one or more hydrophilic layers between the adhesive layer and the active layer.

39. A method according to aspect 38, wherein the one or more hydrophilic layers comprise a dehydrated hydrogel.

40. A method according to aspect 39, wherein said dehydrated hydrogel is a cross-linked poly(acrylic acid).

41. A method according to aspect 39, wherein said dehydrated hydrogel is a hydroxyether cellulose.

42. A method for delaying delivery of an active from an active layer disposed in a transdermal or transmucosal dosage form to a subject's skin or mucosa, said method comprising:
disposing, in the dosage form, one or more hydrophilic layers between the active layer and the subject's skin or mucosa.

43. A method according to aspect 42, wherein the one or more hydrophilic layers comprise a dehydrated hydrogel.

44. A method according to aspect 43, wherein said dehydrated hydrogel is a cross-linked poly(acrylic acid).

45. A method according to aspect 43, wherein said dehydrated hydrogel is a hydroxyether cellulose.

46. A method of manufacturing a transdermal or transmucosal dosage form, said method comprising:
providing a substrate; and
disposing at least one transdermal or transmucosal dosage form layer on the substrate using a printing process.

47. A method according to aspect 46, wherein the substrate comprises a backing layer.

48. A method according to aspect 46, wherein, the substrate comprises an adhesive layer.

49. A method according to aspect 46, wherein the substrate comprises a release liner.

50. A method according to aspect 46, wherein the substrate is release liner and wherein said disposing comprises disposing an adhesive layer on the release liner using a printing process.

51. A method according to aspect 46, wherein said disposing comprises disposing a drug layer on the substrate using a printing process.

52. A method according to aspect 46, wherein said disposing comprises disposing a patterned drug layer on the substrate using a printing process.

53. A method according to aspect 46, wherein the printing process is carried out using single drop dispensers.

54. A method according to aspect 46, wherein the printing process is carried out using multiple drop dispensers.

55. A method according to aspect 46, wherein the printing process is carried out by microembossing.

56. A method according to aspect 46, wherein the printing process is carried out by sequential adsorption of polyelectrolytes.
